(19) United States
(12) Patent Application Publication
(10) Pub. No.: US 2011/0059020 A1
(43) Pub. Date: Mar. 10, 2011

(54) LIPOSOME COMPOSITION, AND DIAGNOSTIC CONTRAST AGENT, THERAPEUTIC ENHANCER, AND PHARMACEUTICAL COMPOSITION USING THE SAME

(75) Inventors: Hiroyuki HIRAI, Ashigarakami-gun (JP); Katsuro Tachibana, Fukuoka-shi (JP)

(73) Assignees: FUJIFILM Corporation, Tokyo (JP); FUKUOKA UNIVERSITY, Fukuoka-shi (JP)

(21) Appl. No.: 12/876,686

(22) Filed: Sep. 7, 2010

(30) Foreign Application Priority Data
Sep. 8, 2009 (JP) 2009-207279

(51) Int. Cl. A61K 49/00; A61K 9/127

(52) U.S. Cl. 424/9.1; 424/450; 977/773; 977/907; 977/915; 977/927

(57) ABSTRACT

To provide a liposome composition, which contains at least one liposome; gas entrapped in the liposome, and at least one metal oxide particle encapsulated in or adsorbed on the liposome, wherein the liposome composition satisfies a ratio B/A of 0.01 to 5, where A is a volume of the gas contained in the liposome on the basis of micro liter, and B is a mass of the at least one metal oxide particle contained in the liposome on the basis of milligram.

### BACKGROUND OF THE INVENTION

1. Field of the Invention

The present invention relates to a liposome composition containing at least one liposome, which entraps gas therein and encapsulates or adsorbs at least one metal oxide particle therein or thereon, as well as relating to a diagnostic contrast agent, therapeutic enhancer, and pharmaceutical composition, all using such liposome composition.

2. Description of the Related Art

In recent years, the studies on decomposition processes of hazardous chemicals such as hormone-disrupting substances, germicidal and/or antibacterial treatment for hazardous microorganisms, and cancer treatment have been conducted using titanium oxide, which is known as a photocatalyst. This attempt uses oxidizability of various active oxygen, such as hydroxyl radicals, and singlet oxygen generated by applying ultraviolet rays having a wavelength of 380 nm or shorter, or ultrasonic waves. Especially, the ultrasonic radiation has a characteristic that it has a large permeation (affecting) distance in a water phase compared to the UV radiation, and there is a small influence to normal cells. Therefore, applications thereof in various fields have been expected (see, for example, R. Cai, Y. Kubota, T. Shuin, et al., Cancer Res. 52 (1992) 2346-2348., Japanese Patent Application Laid-Open (JP-A) Nos. 2008-195653 and 2006-150345, and Japanese Patent (JP-B) No. 4169078). Moreover, there are also reports saying that other than titanium oxide, semiconductor particles such as tin oxide or zinc oxide have the same effect (see, for example, JP-B No. 4103929).

The ultrasonic therapy for cancer or the like includes those using heat generated due to ultrasonic absorption by biotissues, those using mechanical functions of ultrasonic vibration, and a sonochemistry therapy in which a chemical reaction of a compound administered in a living body is induced by using a cavitation effect initiated by ultrasonic waves. There are various reports such that an application of ultrasonic waves to cancer cells leads apoptosis to thereby inhibit a growth of the cancer cells (see, for example, Q. Liu, X. Wang, P. Wang, et al., Ultrasonics (2006), 45, 56-60, H. Honda, Q. L. Zhao, T. Kondo, “Ultrasound” in Med. & Biol. 28 (2002) 673-682, JP-A No. 11-92360).

In the case where inorganic particles are applied in vivo as a medical material, because of their insufficient dispersion stability under neutral or approximately neutral physiological conditions, the particles may cause aggregations. Therefore, it is difficult to secure sufficient flowability in blood. For this reason, it is a current situation that an inorganic particle dispersion liquid cannot be directly administered in a blood vessel as an injection.

Meanwhile, a liposome has been attempted to use for carrying particles into cells. The liposome is a vesicle formed of lipids that are also constitutional substances of a biological membrane, and has excellent compatibility to living bodies. In addition, it is possible to encapsulate various medicines in the vesicle. Therefore, the liposome has been widely used as a carrier for medicines. Moreover, since specificity to a cell or tissue can be provided to the liposome by changing the polarity, particle diameter or used lipid substances of the liposome, or bonding a specific ligand (e.g. an antigen, antibody, and sugar), the liposome has been attracted great attention as a drug carrier capable of targeting, and has been clinically applied as a carrier of a chemotherapeutic agent having a strong side effect, such as an anticancer agent (see, for example, JP-A Nos. 05-58879, 2000-319165, and 2006-273740).

However, it is expected that a therapeutic effect obtainable by ultrasonic radiation reduces as particles are encapsulated in the liposome.

Recently, an ultrasonic contrast agent (SONAZOID, manufactured by Daiichi Sankyo Company, Limited) in which perflubutane (i.e. inert gas) is encapsulated in a liposome has been put on the market, but therapeutic use thereof has not been approved yet.

Moreover, it has been proposed a method in which a gas precursor which will be activated depending on a temperature is encapsulated in a liposome, and image diagnoses or heat treatments are carried out by using an increase of the temperature due to ultrasonic radiation to such liposome (see, for example, U.S. Pat. No. 7,078,015).

Although this method is simple and easy, the method has a dangerous possibility such that rapidly induced heat may damage the entire tissue.

Accordingly, it is the current situation that there is a strong demand for the immediate development of a liposome composition, which is excellent is dispersion stability under the approximately neutral physiological conditions, and is applicable for a diagnostic contrast agent, a therapeutic enhancer, and a pharmaceutical composition.

### BRIEF SUMMARY OF THE INVENTION

The present invention aims at solving various problems in the art and achieving the following object. Namely, an object of the present invention is to provide a liposome composition, which is excellent is dispersion stability under neutral or approximately neutral physiological conditions, and is applicable for a diagnostic contrast agent, a therapeutic enhancer, and a pharmaceutical composition, as well as providing a diagnostic contrast agent, a therapeutic enhancer, and a pharmaceutical composition, all using such liposome composition.

As a result of diligent studies and researches conducted by the present inventors, they have reached the following insight. That is, a liposome composition which contains at least one liposome entrapping gas therein, and encapsulating or adsorbing metal oxide particles therein or thereon, where a volume (μL) of the gas (A) contained in the liposome and a mass (mg) of the metal oxide particles (B) contained in the liposome have a ratio B/A of 0.01 to 5, is excellent in dispersion stability under neutral or approximately neutral physiological conditions, and is applicable for a diagnostic contrast agent, a therapeutic enhancer, and a pharmaceutical composition.

The present invention is based upon the insight of the present inventors, and means for solving the aforementioned problems are as follows.

<1> A liposome composition, containing:

at least one liposome;

gas entrapped in the liposome; and

at least one metal oxide particle encapsulated in or adsorbed on the liposome,

wherein the liposome composition satisfies a ratio B/A of 0.01 to 5, where A is a volume of the gas contained in the liposome on the basis of micro liter, and B is a mass of the at least one metal oxide particle contained in the liposome on the basis of milligram.

<2> The liposome composition according to <1>, wherein the liposome composition has a volume average dispersed-particle diameter of 20 nm to 20 μm.
<3> The liposome composition according to any of <1> or <2>, wherein the gas is at least one selected from the group consisting of oxygen, nitrogen, carbon dioxide, xenon, krypton, argon, hydrofluorocarbons, and perfluorocarbons.
<4> The liposome composition according to any one of <1> to <3>, wherein the at least one metal oxide particle has a volume average particle diameter of 1 nm to 50 nm.
<5> The liposome composition according to any one of <1> to <4>, wherein the metal oxide particle is a particle of metal oxide, which is at least one selected from the group consisting of titanium oxide, zinc oxide, iron oxide, tin oxide, and zirconium oxide.
<6> The liposome composition according to any one of <1> to <5>, further containing a receptor bonded to or contained in the liposome, wherein the receptor is capable of specifically recognizing a certain tissue.
<7> The liposome composition according to any one of <1> to <6>, wherein the liposome composition is ultrasonic sensitive.
<8> The liposome composition according to any one of <1> to <7>, wherein the liposome composition is used for medical purposes.
<9> A diagnostic contrast agent containing the liposome composition as defined in any one of <1> to <8>.
<10> A therapeutic enhancer containing the liposome composition as defined in any one of <1> to <8>.
<11> A pharmaceutical composition containing the liposome composition as defined in any one of <1> to <8>.
<12> A diagnose method, containing administering the diagnostic contrast agent as defined in <9> to a body.
<13> A method for enhancing a therapy, containing administering the therapeutic enhancer as defined in <10> to a body.
<14> A therapeutic method, containing administering the pharmaceutical composition as defined in <11> to a body.

The present invention contributes to solve various problems in the art, and provides a liposome composition, which is excellent is dispersion stability under neutral or approximately neutral physiological conditions, and is applicable for a diagnostic contrast agent, a therapeutic enhancer, and a pharmaceutical composition, as well as providing a diagnostic contrast agent, a therapeutic enhancer, and a pharmaceutical composition, all using such liposome composition.

### BRIEF DESCRIPTION OF THE DRAWINGS

FIG. 1 is a schematic diagram showing, as one embodiment of the present invention, a liposome composition containing a liposome which entraps gas therein, and adsorbs metal oxide particles. In FIG. 1, “11” is a liposome composition, “17” is a liposome, “12” is a hydrophilic part, “13” is a hydrophobic part, “14” is a metal oxide particle (a surface of which may be modified with a hydrophilic compound), “15” is gas (which may be covered with a lipid), and “16” is a receptor.

FIG. 2 is a schematic diagram showing, as another embodiment of the present invention, showing a liposome composition containing a liposome which contains gas therein, and encapsulates metal oxide particles. In FIG. 2, “21” is a liposome composition, “27” is a liposome, “22” is a hydrophilic part, “23” is a hydrophobic part, “24” is a metal oxide particle (a surface of which may be modified with a hydrophilic compound), “25” is gas (which may be covered with a lipid), and “26” is a receptor.

FIG. 3A is a schematic diagram showing one example of the positioning of the liposome, metal oxide particles, and gas in the liposome composition of the present invention. In FIG. 3A, “31” is a liposome composition, “36” is a liposome, “32” is a metal oxide particle, “33” is an aqueous solution, and “34” is gas.

FIG. 3B is a schematic diagram showing another example of the positioning of the liposome, metal oxide particles, and gas in the liposome composition of the present invention. In FIG. 3B, “31” is a liposome composition, “36” is a liposome, “32” is a metal oxide particle, and “34” is gas.

FIG. 3C is a schematic diagram showing another example of the positioning of the liposome, metal oxide particles, and gas in the liposome composition of the present invention. In FIG. 3C, “31” is a liposome composition, “36” is a liposome, “32” is a metal oxide particle, and “34” is gas.

FIG. 3D is a schematic diagram showing another example of the positioning of the liposome, metal oxide particles, and gas in the liposome composition of the present invention. In FIG. 3D, “31” is a liposome composition, “36” is a liposome, “32” is a metal oxide particle, and “34” is gas.

FIG. 3E is a schematic diagram showing another example of the positioning of the liposome, metal oxide particles, and gas in the liposome composition of the present invention. In FIG. 3E, “31” is a liposome composition, “36” is a liposome, “32” is a metal oxide particle, and “34” is gas.

### DETAILED DESCRIPTION OF THE INVENTION

### Liposome Composition

The liposome composition of the present invention contains at least one liposome, gas entrapped in the liposome, and at least one metal oxide particle encapsulated in or adsorbed onto the liposome, and may further contain other substances, if necessary.

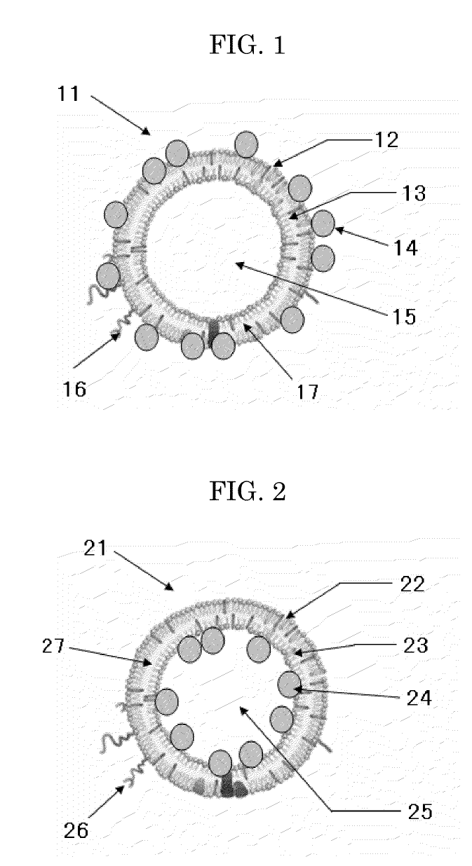

Embodiments of the liposome composition will be explained with reference to FIGS. 1 to 3E.

FIG. 1 is a schematic diagram showing, as one embodiment of the present invention, a liposome composition containing a liposome which contains gas in the liposome and adsorbs metal oxide particles on the liposome. In FIG. 1, the gas is contained in the space present in the center part of the liposome, and the metal oxide particle is adsorbed by the hydrophilic part of the liposome. In addition, a receptor is bonded to the liposome.

FIG. 2 is a schematic diagram showing, as another embodiment of the present invention, a liposome composition containing a liposome which entraps gas and encapsulates metal oxide particles in the liposome. In FIG. 2, the gas is contained in the space present in the center part of the liposome, and the metal oxide particle is encapsulated in the liposome. In addition, a receptor is bonded to the liposome.

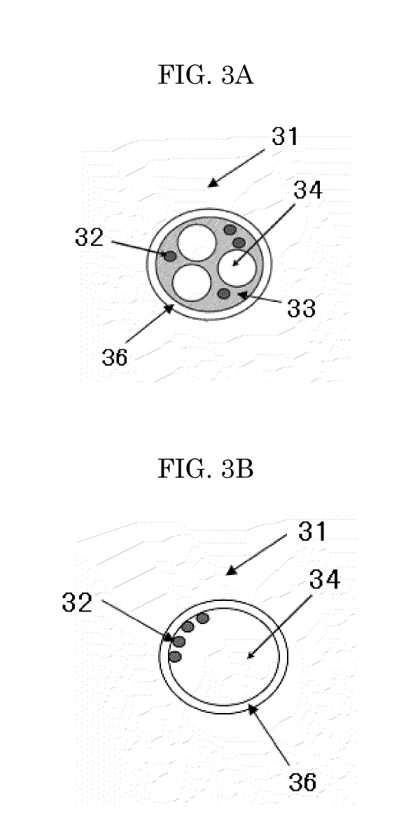

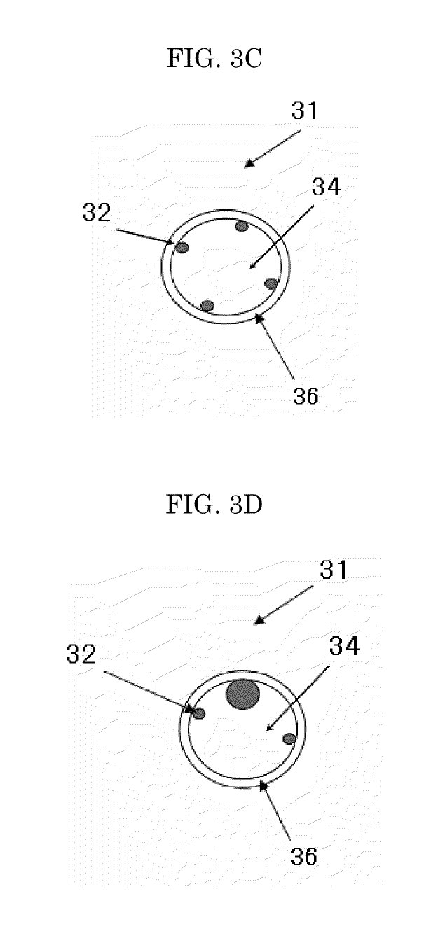

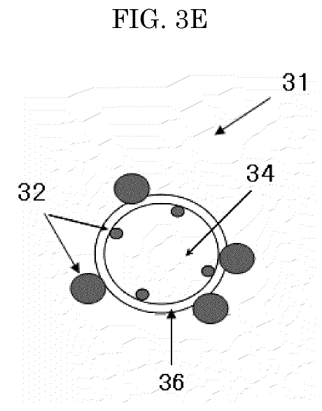

FIGS. 3A to 3E are schematic diagrams showing examples of the positioning of the liposome, metal oxide particles, and gas in the liposome composition of the present invention. In FIG. 3A, the gas coated with a lipid and the metal oxide particles are present in the center part of the liposome, and the rest of the center part is filled with an aqueous solution. In each of FIGS. 3B to 3D, the gas is present in the space at the center part of the liposome, and the metal oxide particles are encapsulated in the liposome. In FIG. 3E, the gas is present in the space at the center part of the liposome, and metal oxide particles are encapsulated in and adsorbed on the liposome.

The gas may be covered with a lipid. Moreover, the gas may be present in the hydrophobic part of the liposome.

The metal oxide particle(s) may be encapsulated and present in the space at the center part of the liposome. When a plurality of the metal oxide particles are contained in the liposome composition, the size of the metal oxide particles may be different to each other.

The liposome composition of the present invention may be formed of a single layer membrane or multilayer membrane containing two or more layers. Moreover, the lipid covering the gas may be made out of the same or different lipid for forming the liposome.

In the liposome composition of the present invention, the liposome entrapping the gas therein and the metal oxide particle(s) are each present to have a distance with which an interaction between the liposome and the metal oxide particle(s) can be initiated by ultrasonic radiation.

The interaction is for example to exhibit a synergistic effect by superimposing the regain where the gas exhibits a cavitation effect by adsorbing ultrasonic waves, and the region where active oxygen generated by the metal oxide particle(s) is present.

### <Ratio B/A>

The liposome composition of the present invention which contains at least one liposome entrapping gas therein and encapsulating or adsorbing at least one metal oxide particle therein or thereon needs to be stable under neutral physiological conditions in vivo. A liposome composition which contains only gas tends to float in a body fluid such as blood, and a liposome composition which contains only at least one metal oxide particle tends to precipitate in a body fluid such as blood. Therefore, it is necessary for the liposome composition to have a balance between buoyancy and gravity so as to reach an affected part such as cancer cells.

To this end, a ratio B/A of a mass (mg) of the metal oxide particle(s) (B) contained in the liposome to a volume (μL) of the gas (A) contained in the liposome is suitably selected depending on the density of the gas and the metal oxide particle(s), provided that it is 0.01 to 5, but it is preferably 0.05 to 3, more preferably 0.5 to 2. When the ratio B/A is less than 0.01, an effect of killing cancer cells or the like obtainable by using the combination of the gas and the metal oxide particles reduces. When the ratio B/A is more than 5, the stability of the liposome composition is insufficient, and thus the liposome composition tends to cause separation or precipitation under physiological conditions. On the other hand, when the ratio B/A is in the aforementioned preferable range, it is advantageous because the obtainable effect of killing cancer cells or the like is significantly enhanced, and also the liposome composition stably present in a body fluid such as blood. When the liposome composition stably present in blood, the liposome composition is easily conveyed by a blood flow, which makes the liposome composition easily reach the cancer cells, and increases anticancer activities. In addition, it increases a contrasting effect, which makes diagnosis easy.

It is preferred that the amount of the metal oxide particle(s) be larger in the liposome as the amount of the gas is larger.

“Physiological conditions” means that it is in phosphate buffered saline (composition: 137 mM-NaCl, 9.0 mM-Na₂HPO₄, 2.9 mM-NaH₂PO₄) having a pH value of 7.2 to 7.4, at 25° C., and 1 atm.

The reason is not clear why the effect in diagnoses and treatments increases when the gas and the metal oxide particle(s) are used in combination, compared to the case where either of them is used independently. However, it is probably because the metal oxide particle(s) moves more intensely with assistance of buoyancy of the gas, for example by ultrasonic radiation, in the closed space like the liposome. Compared to the case of a liposome itself or a bubble liposome containing gas, it is assumed that the liposome composition of the present invention has an effect of changing resonance frequency of babbles (i.e., the gas) due to a difference in the density between the metal oxide particle(s) and the liposome, which makes a contrast in resulting images large, and makes ultrasonic diagnosis more effective. Moreover, it is also assumed that some kind of effects may be exhibited as the metal oxide particle(s) physically destroy the liposome by ultrasonic radiation, so that the gas directly works on cells of the affected part.

Examples of the positioning of the liposome, metal oxide particle(s) and gas are shown in FIGS. 3A to 3E. It is also a preferable embodiment such that two types of the metal oxide particles, namely, the metal oxide particle(s) of a large size (e.g., about 20 nm) and the metal oxide particle(s) of a small size (e.g., about 5 nm), are provided, and the metal oxide particle(s) of the large size is adsorbed on the liposome, and the metal oxide particle(s) of the large size is encapsulated in the liposome. By using the aforementioned technique, the ultrasonic sensitivity of the liposome composition can be enhanced.

### <Volume Average Dispersed-Particle Diameter>

The volume average dispersed-particle diameter of the liposome composition, which containing at least one liposome entrapping the gas therein and encapsulating or adsorbing at least one metal oxide particle, is suitably selected depending on the intended purpose without any restriction. The volume average dispersed-particle diameter thereof is preferably 20 nm to 20 μm, and more preferably 50 nm to 10 μm. When the volume average dispersed-particle diameter thereof is less than 20 nm, it is difficult to synthesize a liposome itself, and is also difficult to stably contain the gas or metal oxide particle(s) in the liposome. When the volume average dispersed-particle diameter thereof is more than 20 μm, vascular occlusion or hematogenous disorder may occur in capillary vessels or a part of a vessel where a blood flow is slow, and the liposome composition may not readily reach an affected part, such as cancer cells. When the volume average dispersed-particle diameter thereof is in the aforementioned preferable range, on the other hand, sufficient dispersion stability and fluidity can be attained in a solution such as a blood stream so that such liposome can be used for medical purposes such as diagnoses and treatments. Therefore, the liposome composition with such volume average dispersed-particle diameter is advantageous.

In the case where the liposome composition is used as a diagnostic contrast agent, the volume average dispersed-particle diameter of the liposome composition is suitably selected depending on the intended purpose without any restriction, but it is preferably 100 nm to 20 μm, more preferably 1 μm to 10 μm. When the volume average dispersed-particle diameter thereof in the more preferable range, it is advantageous because the liposome composition tends to provide a clear contrast in a resulting image.

In the case where the liposome composition is used as a therapeutic enhancer, the volume average dispersed-particle diameter thereof is suitably selected depending on the intended purpose without any restriction. For example, in case of a cancer treatment, it is preferably 50 nm to 500 nm, more preferably 60 nm to 300 nm. When the volume average dispersed-particle diameter thereof is in the more preferable range, it is possible to preferentially accumulate such liposome composition onto cancer tissues due to an enhanced permeation and retention effect (EPR effect), and thus it is effective in the enhancement of the cancer treatment.

The volume average dispersed-particle diameter of the liposome composition can be measured by dynamic light scattering. For example, it can be measured by means of a microtrack UPA-UT151 particle size distribution analyzer (manufactured by Nikkiso Co., Ltd.).

### <Ultrasonic Sensitivity>

The liposome composition is preferably ultrasonic sensitive, as it will provide the liposome composition with a therapeutic effect or diagnostic effect for cancer or the like.

Being ultrasonic sensitive means that the liposome composition is heated, receives mechanical vibrations, or exhibits a cavitation effect by ultrasonic radiation.

By applying ultrasonic waves to the liposome composition containing at least one liposome in which the gas and the metal oxide particle(s) are both present, the obtainable effect (e.g. a bactericidal effect in dental treatments, and an effect of killing or damaging cancer cells) significantly improves.

### <Gas>

The gas is suitably selected depending on the intended purpose without any restriction, provided that it can be entrapped in the liposome. The gas is preferably selected from those being present as a vapor under physiological conditions.

“Physiological conditions” are as mentioned above.

Examples of the preferable gas include oxygen, nitrogen, carbon dioxide, xenon, krypton, argon, hydrofluorocarbons, and perfluorocarbons. These may be used independently, or in combination.

Among them, xenon, krypton, argon, hydrofluorocarbons, and perfluorocarbons are advantageously used. This is because these are insoluble in water, and molecular size and density thereof are large so that these can be stably contained within the liposome, which leads high sensitivity for diagnoses, and high therapeutic effect.

Examples of the hydrofluorocarbons include 1,1,1,2,2-pentafluoroethane, 1,1,2,2-tetrafluoroethane, 1,1,1-trifluoroethane, 1,1-difluoroethane, 1,1,1,2,3,3,3-heptafluoropropane, 1,1,1,3,3,3-hexafluoropropane, 1,1,2,2,3-pentafluoropropane, and 1,1,1,2,3,4,4,5,5,5-decafluoropentane.

Examples of the perfluorocarbons include those known as ultrasonic contrast agents, such as perfluoroethane, perfluoropropane, perfluorobutane, perfluorocyclobutane, perfluoropentane, and hexafluoro-1,3-butadiene.

The amount of the gas contained in the liposome composition is suitably selected depending on the intended purpose without any restriction, provided that it is equal to or smaller than the volume of the void(s) of the liposome composition. The amount of the gas is preferably 10% to 100%, more preferably 20% to 95%, and even more preferably 25% to 90% relative to the volume of the void(s) of the liposome composition. When the amount of the gas is less than 10%, the obtainable therapeutic effect is small. When the amount thereof is more than 100%, the condition of the liposome composition becomes unstable. On the other hand, when the amount of the gas contained in the liposome composition is in the aforementioned even more preferable range, it is advantageous, as sensitivity for diagnoses increases, and a significant effect of enhancing treatments can be attained.

The amount of the gas contained in the liposome composition can be assumed, for example, by obtaining an amount of the gas by gas chromatography or the like, and comparing the obtained value with the size of the liposome composition measured by an optical microscope or electron microscope.

Moreover, an amount of the gas contained in a dispersion liquid, in which the liposome composition of the present invention is dispersed, is suitably selected depending on the intended purpose without any restriction. The amount thereof is preferably 0.1 μL to 100 μL relative to 1 mL of the dispersion liquid. When the amount of the gas in the dispersion liquid in which the liposome composition is dispersed is less than 0.1 μL, an effect of diagnoses and an effect of enhancing treatments are not obtained. In this case, moreover, a pharmacological agent cannot be administered in a uniform concentration because the liposome composition containing the metal oxide particle(s) is precipitated in a storage container, which may cause a significant accident. When the amount of the gas is more than 100 μL, the dispersion liquid is unstable so that the liposome composition is floated in the container. Therefore, a pharmacological agent cannot be administered in a uniform concentration, which may cause a significant accident. On the other hand, when the amount of the gas contained in the dispersion liquid in which the liposome composition is dispersed is within the aforementioned preferable range, it is advantageous because the liposome composition is stably present so that sensitivity for diagnoses increases and a significant effect of enhancing treatments can be attained.

The gas may be covered with a lipid. Moreover, the gas may be present in the hydrophobic part of the liposome.

### <Metal Oxide Particle>

The metal oxide particle(s) is suitably selected depending on the intended purpose without any restriction, but those having low toxicity in vivo are preferable. Since the liposome composition of the present invention contains the liposome encapsulating or adsorbing the at metal oxide particle(s) therein or thereon, various mechanisms of action caused by ultrasonic radiation can be used.

Examples of the metal oxide particle(s) include a particle(s) of metal oxides such as titanium oxide (TiO₂), zinc oxide (ZnO), tin oxide (SnO₂), iron oxide (e.g. magnetite, and Fe₂O₃), ferrite (e.g., zinc ferrite, magnesium ferrite, barium ferrite, and magnesium ferrite), zirconium oxide (ZrO₂), WO₃, MoO₃, Al₂O₃, Y₂O₃, and La₂O₃. These may be used independently or in combination.

Among them, a titanium oxide particle(s), zinc oxide particle(s), tin oxide particle(s), iron oxide particle(s), and zirconium oxide particle(s) are preferable. Moreover, an anatase or rutile titanium oxide particle(s) is more preferable because it contributes to generate a large amount of active oxygen, and has excellent effect of killing cancer cells. Furthermore, a zinc oxide particle(s) and iron oxide particle(s) are more preferable because they contain essential elements for organisms.

The shape of the metal oxide particle(s) is suitably selected depending on the intended purpose without any restriction. Preferable examples thereof include a spherical shape, cubic shape, and oval shape.

The formation method of the metal oxide particle(s) is suitably selected from methods known in the art depending on the intended purpose. Examples thereof include a gas phase method, a liquid phase method, and other known methods for forming nanoparticles. Among them, the liquid phase method is preferable because it has excellent mass productivity.

A solvent for use in the liquid phase method is suitably selected depending on the intended purpose without any restriction. Examples thereof include an organic solvent, water, and a mixed solution of an organic solvent and water. Among them, water and a hydrophilic solvent are preferable.

The volume average particle diameter of the metal oxide particles is suitably selected depending on the intended purpose without any restriction, but it is preferably 1 nm to 50 nm, more preferably 2 nm to 20 nm. When the volume average particle diameter of the metal oxide particles is less than 1 nm, each particle itself tends to be unstable. When the volume average particle diameter of the metal oxide particles is more than 50 nm, sedimentation thereof tends to occur, and it is difficult to introduce the metal oxide particles of such size into the liposome. On the other hand, when the volume average particle diameter of the metal oxide particles is within the aforementioned more preferable range, it is advantageous because the resulting liposome composition can provide a large effect of enhancing treatments.

A transmittance electron microscope (TEM) can be used for determination of the volume average particle diameter.

The volume average particle diameter means a diameter of a circle which is determined to have the same area to that of the image of the metal oxide particle taken by an electron microscopic photography.

Generally, the metal oxide particles tend to aggregate to each other at around the isoelectric point, and thus it is difficult to introduce the metal oxide particles into the liposome under such condition. Therefore, the pH value of the dispersion liquid is adjusted so that zeta-potential at a surface of the particle is sifted to positive or negative for stabilizing, and then the metal oxide particles are introduced into the liposome. Alternatively, the metal oxide particles may be subjected to a hydrophilication treatment by making each surface of the metal oxide particles adsorb a surfactant, and then introduced into the liposome.

The amount of the metal oxide particle(s) relative to the total amount of lipids in the liposome is suitably selected depending on the intended purpose without any restriction, but it is preferably 0.1% to 50,000%, more preferably 1% to 10,000% based on a mass ratio {(metal oxide particles/the total lipids of the liposome)×100}. When the amount of the metal oxide particle(s) is less than 0.1%, the synergistic effect due to the combination of the gas and the metal oxide particle may not be attained. When the amount thereof is more than 50,000%, the resulting liposome composition may become unstable, and sedimentation thereof may be occur under the physiological conditions. On the other hand, when the amount thereof is in the aforementioned more preferable range, it is advantageous because the resulting liposome composition is stable under the physiological conditions, and provided a sufficient effect of enhancing treatments.

The metal oxide particle(s) may be encapsulated in, or adsorbed on the liposome, or may be both.

Especially in the case where the liposome composition is used as a therapeutic enhancer or pharmaceutical composition, an embodiment in which the metal oxide particle(s) is adsorbed on the outer side of the liposome is preferable for the following reason. For example, when by-products such as hydroxyl radicals and singlet oxygen are utilized, the generated hydroxyl radicals or singlet oxygen is shielded by the wall of the liposome so that the aforementioned active oxygen can easily and directly effect on an affected part by ultrasonic radiation. Accordingly, an effect of enhancing treatments can be attained.

In the case where a cavitation effect or mechanical function is expected, an embodiment in which the metal oxide particle(s) is encapsulated in the liposome is preferable because the liposome is expected to break to open due to sonoporation to thereby attaining an effect of enhancing treatments.

### <Liposome>

The liposome for used in the liposome composition of the present invention, which includes the gas therein and encapsulates or adsorbs the metal oxide particle(s) therein or thereon, is a closed vesicle containing a neutral lipid, and a negatively-charged lipid and/or a positively-charged compound. The lipid may be further bonded with a nonionic water-soluble polymer or protein.

The neutral lipid is a lipid having cations and anions in the equivalent numbers in a physiologic pH aqueous medium, namely an aqueous medium having a pH value of 6.5 to 7.5.

The neutral lipid is suitably selected depending on the intended purpose without any restriction. Examples thereof include: phosphatidic acid derivatives such as dipalmitoylphosphatidylcholine, and phosphatidylethanol amine; glycolipids such as digalactosyl glyceride, and galactosyl glyceride; sphingosine derivatives such as sphingomyelin; and sterols such as cholesterol, ergosterol, and lanosterol. These may be used independently or in combination.

Among them, the phosphatidic acid derivatives, glycolipids, and sterols are preferable, the phosphatidic acid derivatives and sterols are more preferable, and the phosphatidic acid derivatives are even more preferable.

Among the phosphatidic acid derivatives, di(C10-22 alkanoyl or alkenoyl) phosphatidylcholine derivatives are preferable, and dipalmitoylphosphatidylcholine, and distearoyl-sn-glycero-phosphatidylcholine are more preferable.

Examples of the aforementioned C10-22 alkanoyl or alkenoyl group include a decylyl group, an undecylyl group, a dodecylyl group, a tridecylyl group, a tetradecylyl group, a pentadecylyl group, a hexadecylyl group, a heptadecylyl group, an octadecylyl group, a nonadecylyl group, an icosyl group, a henicosyl group, a docosyl group, a decenyl group, a dodecenyl group, a tetradecenyl group, a hexadecenyl group, an octadecenyl group, an icocenyl group, and a dococenyl group.

The aforementioned “di(C10-22 alkanoyl or alkenoyl)” means that two hydroxyl groups contained in phosphatidylcholine are each esterification-bonded to a carboxylic acid of the C10-22 alkanoyl or alkenoyl group.

The sterols such as cholesterol themselves can be used as a constitutional component of the liposome, they ma be used, if necessary, added to other neutral lipids.

The negatively-charged lipid is a lipid having more cations than anions in a physiologic pH aqueous medium.

The negatively-charged lipid is suitably selected depending on the intended purpose without any restriction. Examples thereof include hydrogenated egg phosphatidylserine sodium salt; phosphatidylglycerols such as dipalmitoylphosphatidylglycerol; phosphatidylserines such as dipalmitoylphosphatidylserine; and phosphatidylinositols such as dipalmitoylphosphatidylinositol. These may be used independently or in combination.

Among them, phosphatidylglycerols are preferable, and dipalmitoylphosphatidylglycerol is more preferable.

The positively-charged compound is a compound having more anions than cations in a physiologic pH aqueous medium.

The positively-charged compound is suitably selected depending on the intended purpose without any restriction. Examples thereof include a positively-charged lipid, a cationic surfactant, and a cationic water-soluble polymer. These may be used independently or in combination.

The positively-charged lipid is suitably selected depending on the intended purpose without any restriction. Examples thereof include: chain hydrocarbon amines such as stearyl amine, and oleyl amine; amine derivatives of cholesterol such as 3-β[N-(N′,N′-dimethylaminoethane)carbamoyl] cholesterol; N-α-trimethylammonioacetyl di(C10-20 alkyl or alkenyl)-D-glutamate chlorides such as N-α-trimethylammonioacetyldidodecyl-D-glutamate chloride; and N-[1-(2,3-di(C10-20 alkyl or alkenyl)oxy)propyl]-N,N,N-trimethylammonium chlorides such as N-[1-(2,3-dioleyloxy)propyl]-N,N,N-trimethylammonium chloride.

Examples of the alkyl group include a pentyl group, a hexyl group, an octyl group, a nonyl group, a decyl group, a dodecyl group, a tetradecyl group, a hexadecyl group, an octadecyl group, an icosyl group, a docosyl group, a tetracosyl group, a hexacosyl group, an octacosyl group, and a triacontasyl group. Among them, C5-30 alkyl groups are preferable, and C10-20 alkyl groups are more preferable.

Examples of C10-20 alkyl or alkenyl group include a decyl group, an undecyl group, a dodecyl group, a tridecyl group, a tetradecyl group, a pentadecyl group, a hexadecyl group, a heptadecyl group, an octadecyl, a nonadecyl group, an icosyl group, a decenyl group, a decynyl group, an undecynyl group, a dodecynyl group, and a tridecynyl group.

Among these positively-charged lipids, alkyl amine, N-α-trimethylammonioacetyl di(C10-20 alkyl or alkenyl)-D-glutamate chloride are preferable, and N-α-trimethylammonioacetyldidodecyl-D-glutamate chloride is more preferable.

The cationic surfactant is suitably selected cationic surfactants known in the art without any restriction. Examples thereof include cationic surfactants disclosed in M. J. ROSEN, (Tsubone, Sakamoto, trans.), Surfactants and Interfacial Phenomena (Fragrance Journal Ltd., 1995), pp. 16-20. The cationic surfactant may be used independently or in combination.

Among the cationic surfactants, long-chain alkyl amine and salts thereof, long-chain alkyl or aralkyl quaternary ammonium salt, polyoxyethylene adduct of long-chain alkyl amine or salts thereof, polyoxyethylene adduct of long-chain alkyl quaternary ammonium salt, and long-chain alkyl amine oxide are preferable, the long-chain alkyl amine or salts thereof, long-chain alkyl or alkenyl quaternary ammonium salt, and polyoxyethylene adduct of long-chain alkyl amine or salts thereof are more preferable, and the long-chain alkyl amine or salts thereof is even more preferable.

A highly concentrated cationic surfactant may destroy the liposome, but a cationic surfactant can be contained in the liposome as a component, if it is in a small amount (see Urbaneja et al., Biochem. J, vol. 270, pp. 305-308, 1990). Accordingly, by adding an amount of the cationic surfactant, which will not adversely affect the formation of the liposome, or which will not destroy the formed liposome, or adding the cationic surfactant in a dispersion liquid in which the previously formed liposome is dispersed to adsorb the cationic surfactant on the surface of the liposome, the cationic surfactant can be present as a component of the liposome to reduce the negatively-charged state of the polymer-modified liposome. This is preferable because the toxicity to living bodies can be reduced.

The cationic water-soluble polymer is suitably selected from cationic water-soluble polymers known in the art without any restriction. Examples thereof include cationic water-soluble polymers disclosed in G. Allen et al., edit., Comprehensive polymer science, (Pergamon Press, 1989) vol. 6. The cationic water-soluble polymer may be used independently or in combination.

Among the aforementioned cationic water-soluble polymers, cationic water-soluble vinyl synthesized polymer, cationic water-soluble polyamino acid, cationic water-soluble synthesized polypeptide, cationic water-soluble natural polymer, and cationic water-soluble modified natural polymer are preferable, the cationic water-soluble vinyl synthesized polymer, cationic water-soluble polyamino acid, and cationic water-soluble synthesized polypeptide are more preferable, and the cationic water-soluble vinyl synthesized polymer is even more preferable.

The manner of the absorption of these cationic water-soluble polymers onto the liposome is different from the manner of absorption of a low-molecular weight compound thereto. The absorption of the polymer to a surface of a solid is stable, and irreversible (see G. Allen et al., edit., Comprehensive polymer science, (Pergamon Press, 1989), vol. 2, pp. 733-754. Accordingly, by adsorbing the cationic water-soluble polymer onto the negatively charged liposome, the negative charges of the liposome reduce. It is preferable because the toxicity to living bodies can be reduced.

In the present invention, each lipid may be bonded to a nonionic water-soluble polymer.

The nonionic water-soluble polymer is suitably selected depending on the intended purpose without any restriction, but preferable examples thereof include: nonionic polyether such as polyethylene glycol; nonionic monoalkoxy polyether such as monomethoxy polyethylene glycol, and monoethoxy polyethylene glycol; nonionic polyamino acid; and nonionic synthesized polypeptide.

The weight average molecular weight of the nonionic water-soluble polymer is suitably selected depending on the intended purpose without any restriction, but it is preferably 1,000 to 12,000, more preferably 1,000 to 5,000.

The diameter of the liposome (i.e. the liposome before including the gas therein) is suitably selected depending on the intended purpose without any restriction. Although the diameter thereof is different depending on how the size of the liposome is controlled, the volume average particle diameter of the liposome is preferably 10 nm to 500 nm, more preferably 20 nm to 200 nm, and even more preferably 20 nm to 100 nm.

Here, the volume average particle diameter means an average value of the particle diameters calculated from the average volume of a plurality of particles, and is calculated by means of a particle size analyzer in accordance with methods known in the art (e.g., R. R. C. New, edit., Liposomes: a practical approach (IRL Press, 1989), pp. 154-160).

### <Other Substances>

Other substances may be suitably selected depending on the intended purpose without any restriction, provided that they do not adversely affect the obtainable effect of the present invention. Examples thereof include a receptor.

### —Receptor—

It is preferable that the liposome composition of the present invention be bonded to or contain a receptor capable of specifically recognizing a certain tissue, because it is effective in diagnoses or treatments for tumors by ultrasonic waves, and it exhibits an effect of instructing killer cells.

The receptor is suitably selected depending on the intended purpose without any restriction. Examples thereof include various receptors that are accumulated specific to abnormal cells such as tumors. These may be used independently, or in combination.

Specific examples of the receptor include various monoclonal antibodies, various proteins, polypeptides, steroids, and immunity-related agents (e.g. immunocyte reactivation substances, activation substances).

The receptor is bonded to or contained in the liposome via a terminal amino group, hydroxyl group or carboxyl group of the aforementioned lipid, water-soluble polymer, or surfactant.

The receptor may cover the entire surface of the liposome, or part of the surface thereof.

### <Production Method>

The production method of the liposome composition containing at least one liposome which entraps the gas therein, and encapsulates or adsorb at least one metal oxide particle therein or thereon, is suitably selected depending on the intended purpose without any restriction.

One embodiment of the production method thereof will be shown below.

1. A metal oxide particle (average particle diameter: 1 nm to 50 nm) dispersion liquid is prepared.
2. Liposomes are formed by combining two or more lipids. Here, the softness of the liposome membrane may be changed (using the deference in the phase-transition points), or domains each having different softness may be formed two-dimensionally in the membrane (using phase separation phenomenon). These characteristics can be controlled by changing the temperature by externally applying electromagnetic stimuli or ultrasonic stimuli.
3. Liposomes, to which, other than the lipids, a charge-controlling agent, protein, and/or nonionic water-soluble polymer are optionally combined, are prepared. By this, a surface charge of the liposome membrane or molecule permeability is controlled at the same time as reducing tendencies thereof for deposition or aggregation, to thereby improve dispersion stability of the liposome.
4. Joining of the metal oxide particles and the liposomes is accelerated by using electrostatic attraction force of various ions, or adhesive force of protein to produce the liposome composition containing at least one liposome encapsulating or adsorbing at lest one metal oxide particle therein, or thereon.
5. The liposome composition containing at least one liposome encapsulating or adsorbing at least one metal oxide particle therein, or thereon is placed in a container filled with gas, and ultrasonic waves are applied thereto under the pressure to thereby make the gas included in the liposome composition.

In the manner mentioned above, the liposome composition of the present invention can be produced.

The liposome composition of the present invention is suitably used for medical purposes.

For example, in the case where the metal oxide particles used in the liposome composition are super paramagnetic particles such as of iron oxide, the liposome composition can be used for MRI diagnosis as well as ultrasonic diagnosis.

For example, the liposome composition of the present invention can be used for treating various illnesses including cancer by using mechanical actions initiated by ultrasonic radiation, or active oxygen such as singlet oxygen and hydroxyl radicals generated by ultrasonic radiation.

The frequency of the ultrasonic wave for use in the radiation is suitably selected depending on the intended purpose without any restriction, but is preferably about 20 KHz to about 20 MHz, more preferably about 600 KHz to about 3 MHz.

The output of the radiation is suitably selected depending on the purpose without any restriction, but is preferably about 0.1 W/cm² to about 100 W/cm², more preferably about 0.5 W/cm² to about 10 W/cm².

The duty cycle of the ultrasonic wave is suitably selected depending on the intended purpose without any restriction, but is preferably about 1% to about 100%, more preferably about 10% to about 50%.

The duration of the ultrasonic radiation is suitably selected depending on the frequency, and output for use, without any restriction, but is preferably about 5 seconds to about 600 seconds, more preferably about 30 seconds to about 300 seconds.

The liposome composition of the present invention can be effectively used for treatments of various cancers, virus infections, intercellular parasite infections, pulmonary fibrosis, hepatic cirrhosis, chronic nephritis, arteriosclerosis, leukemia, and blood vessel stenosis.

Examples of the cancers include all solid cancers grown on the surface or inner part of organs, such as a lung cancer, liver cancer, pancreatic cancer, gastrointestinal cancer, bladder cancer, renal cancer, and brain tumor. Among them, the liposome composition of the present invention can be effectively used for a treatment of a cancer that is present in the deep part of a body, to which a photo-dynamic therapy cannot be performed.

With regard to other illness, as the focus or infected cell (affected cell) is located in the inner part of the organ, a treatment can be performed by accumulating the liposome composition of the present invention on such part using an appropriate method, and then externally applying ultrasonic waves.

### (Diagnostic Contrast Agent, Therapeutic Enhancer, Pharmaceutical Composition)

### <Diagnostic Contrast Agent>

The diagnostic contrast agent of the present invention contains at least the liposome composition of the invention, and may further contain other substances, if necessary.

The amount of the liposome composition contained in the diagnostic contrast agent is suitably selected depending on the intended purpose without any restriction. The diagnostic contrast agent may be the liposome composition of the present invention, itself.

Other substances are suitably selected, for example, from pharmacologically acceptable carriers, without any restriction. Examples thereof include ethanol, water, starch, saccharides, and dextran. The amount of other substances contained in the diagnostic contrast agent is suitably selected depending on the intended purpose without any restriction, provided that it does not adversely affect the obtainable effect of the liposome composition.

The diagnostic contrast agent may be used independently, or in combination with a medicine containing other substance(s) as an active ingredient. Moreover, the diagnostic contrast agent may be used by being formulated in a medicine containing other substance(s) as an active ingredient.

### <Therapeutic Enhancer>

The therapeutic enhancer of the present invention contains at least the liposome composition of the present invention, and may further contain other substances, if necessary.

The therapeutic enhancement is to exhibit a therapeutic effect of a therapeutic agent such as the liposome composition of the present invention, which has no or significantly small effect as a therapeutic effect when it is used singly, by applying physical energy such as ultrasonic wave, electronic field, or magnetic field, or to attain the increased therapeutic effect by combining physical energy such as ultrasonic waves, electronic field, or magnetic field, though the physical energy itself has no or significantly small therapeutic effect.

The amount of the liposome composition of the present invention contained in the therapeutic enhancer is suitably selected depending on the intended purpose without any restriction. The therapeutic enhancer may be the liposome composition of the present invention, itself.

Other substances are suitably selected, for example, from pharmacologically acceptable carriers, without any restriction. Examples thereof include ethanol, water, starch, saccharides, and dextran. The amount of other substances contained in the therapeutic enhancer is suitably selected depending on the intended purpose without any restriction, provided that it does not adversely affect the obtainable effect of the liposome composition.

The therapeutic enhancer may be used independently, or in combination with a medicine containing other substance(s) as an active ingredient. Moreover, the therapeutic enhancer may be used by being formulated in a medicine containing other substance(s) as an active ingredient.

### <Pharmaceutical Composition>

The pharmaceutical composition of the present invention contains at least the liposome composition of the present invention, and may further contain other substances, if necessary.

The amount of the liposome composition of the present invention contained in the pharmaceutical composition is suitably selected depending on the intended purpose without any restriction. The pharmaceutical composition may be the liposome composition of the present invention, itself.

Other substances are suitably selected, for example, from pharmacologically acceptable carriers, without any restriction. Examples thereof include ethanol, water, starch, saccharides, and dextran. The amount of other substances contained in the pharmaceutical composition is suitably selected depending on the intended purpose without any restriction, provided that it does not adversely affect the obtainable effect of the liposome composition.

The pharmaceutical composition may be used independently, or in combination with a medicine containing other substance(s) as an active ingredient. Moreover, the pharmaceutical composition may be used by being formulated in a medicine containing other substance(s) as an active ingredient.

### —Dosage Form—

The dosage form of the diagnostic contrast agent, therapeutic enhancer, and pharmaceutical composition is suitably selected depending on the intended purpose without any restriction. Examples thereof include parenteral injection (e.g., in vein, in artery, in muscle, subcutis, and intracutaneous), a dispersing agent, and liquids. The diagnostic contrast agent, therapeutic enhancer, and pharmaceutical composition of these dosage forms can be produced in accordance with the conventional methods. In the case where it is administered as parenteral injection, for example, parenteral injection can be attained by formulating the liposome composition of the present invention with various additives generally used for parenterial injection, such as buffer, physiological saline, preservatives, distilled water for injection and the like.

### —Administration—

The administration method of the diagnostic contrast agent, therapeutic enhancer, and pharmaceutical composition is suitably selected depending on the dosage form thereof without any restriction.

The dosage of the diagnostic contrast agent, therapeutic enhancer, and pharmaceutical composition is suitably selected, without any restriction, considering various factors, such as administrating path, age and sex of a patient, and a type and situation of illness. For example, in the case of an adult, it can be administered in an amount of about 0.01 mg/kg to about 10 mg/kg per day, which will be taken at once or separately in a few times.

The period for administering the diagnostic contrast agent, therapeutic enhancer, and pharmaceutical composition is suitably selected depending on the intended purpose without any restriction.

The animal species to be a subject of an administration of the diagnostic contrast agent, therapeutic enhancer, and pharmaceutical composition are suitably selected depending on the intended purpose without any restriction. Examples thereof include humans, monkeys, pigs, cattle, sheep, goats, dogs, cats, mice, rats, and birds.

The liposome composition of the present invention is excellent in dispersion stability in an aqueous solvent in a neutral pH range, and has high diagnostic and therapeutic effect in assistance with ultrasonic waves, as it includes at least one liposome entrapping gas therein, and encapsulating or adsorbing metal oxide particle(s) therein or thereon. Moreover, since the liposome composition of the present invention can accurately visualize the distribution of the gas by a ultrasonic diagnostic equipment, a treatment can be carried out at the same time as highly accurately detecting a lesioned part such as cancer. Therefore, the liposome composition of the present invention contributes to a quality of life (QOL) of a patient.

### EXAMPLES

The present invention will be more specifically explained with Examples hereinafter, but these Examples shall not be construed as limiting the scope of the present invention. Moreover, any modification, which is made in Examples so as not to depart from the meaning of the prior or posterior description, will be included in the technical scope of the present invention.

### Comparative Example 1-1

A solution in which 3 mL of acetic acid was added to 14.2 g of titanium tetraisopropoxide was added to 85 mL of water with sufficient stirring, and the mixture was stirred for 1 hour at room temperature to allow hydrolysis to proceed. Then, to this, 1.3 mL of nitric acid was added, and the mixture was heated to 80° C., and stirred for 6 hours. After cooling the mixture to room temperature, it was filtered through a filter having an opening diameter of 0.45 μm, and the resultant was further subjected to ultrafiltration for desalination. In such manner, a 4% by mass anatase TiO₂ nanoparticle (volume average particle diameter: 6 nm) dispersion liquid having a pH value of 3.5 was obtained. Note that, the volume average particle diameter of the nanoparticles were measured by observing an image through a transmission electron microscope (TEM) (JEM-2000FX, manufactured by JEOL Ltd.).

To COATSOME EL-01-N (containing 54 μmol of L-α-dipalmitoylphosphatidylcholine (DPPC), 40 μmol of cholesterol (CHOL), and 6 μmol of L-α-dipalmitoylphosphatidylglycerol) manufactured by NOF CORPORATION, 2 mL of a liquid in which the aforementioned TiO₂ nanoparticle dispersion liquid was diluted to 0.24% by mass with pure water was added, and then the mixture was vibrated to thereby prepare a weakly negatively-charged liposome composition dispersion liquid (TiO₂ content of 2.4 mg/mL), that was a liposome composition dispersion liquid of Comparative Example 1-1 (hereinafter, may be referred to as Sample 1A).

A volume average dispersed-particle diameter of Sample 1A was measured by means of a microtrack UPA-UT151 particle size analyzer (manufactured by Nikkiso Co., Ltd.), and it was 240 nm.

Sample 1A was stable in a PBS buffer solution (pH 7.2) (under physiological conditions). Note that, the “stable” means the state where no aggregation or precipitation occurs therein after it was left to stand under physiological conditions at 25° C. for 24 hours.

### Example 1-1

Sample 1A obtained in Comparative Example 1-1 was poured into a vial, and the vial was filled with perfluoropropane (PFP) gas. After filling the vial with the gas in the volume that was 1.5 times of the volume of the vial under pressure, ultrasonic waves of 20 kHz and 50 W were applied thereto for 15 minutes. Thereafter, ultrasonic waves of 800 kHz and 30 W were further applied for 60 minutes to thereby obtain a liposome composition dispersion liquid of Example 1-1 (hereinafter, may be referred to as Sample 1B).

A volume average dispersed-particle diameter of Sample 1B was measured in the same manner as in Comparative Example 1-1, and it was 270 nm.

A concentration of the perfluoropropane gas in Sample 1B was determined by a gas chromatograph GC-2014 (manufactured by Shimadzu Corporation), and it was 2.5 μL/mL.

Sample 1B was stable in a PBS buffer solution (pH 7.2).

### Comparative Example 1-2

To COATSOME EL-01-N manufactured by NOF CORPORATION, 2 mL of pure water was added, and the mixture was vibrated to prepare a weakly negatively-charged liposome dispersion liquid (hereinafter, referred to as Sample 1C), that was a liposome dispersion liquid of Comparative Example 1-2 (hereinafter, may be referred to as Sample 1C).

A volume average dispersed-particle diameter of Sample 1C was measured in the same manner as in Comparative Example 1-1, and it was 270 nm.

Sample 1C was stable in a PBS buffer solution (pH 7.2).

### Comparative Example 1-3

Sample 1C obtained in Comparative Example 1-2 was poured into a vial, and the vial was filled with perfluoropropane (PFP) gas. After filling the vial with the gas in the volume that was 1.5 times of the volume of the vial under pressure, ultrasonic waves of 20 kHz and 50 W were applied thereto for 15 minutes. Thereafter, ultrasonic waves of 800 kHz and 30 W were further applied for 60 minutes to thereby obtain a liposome composition dispersion liquid of Comparative Example 1-3 (hereinafter, may be referred to as Sample 1D).

A volume average dispersed-particle diameter of Sample 1D was measured in the same manner as in Comparative Example 1-1, and it was 300 nm.

A concentration of the perfluoropropane gas in Sample 1D was determined in the same manner as in Example 1-1, and it was 2.6 μL/mL.

Sample 1D was stable in a PBS buffer solution (pH 7.2).

### Comparative Example 2-1

In a mixture of 200 mL of methanol and 1.1 mL of water, 3.7 g of zinc acetate was dissolved, and the resulting solution was heated to 60° C. To this solution, a solution in which 2.2 g of KOH was dissolved in 100 mL methanol was added, circulated for 1 hour, and then 250 mL of methanol was removed therefrom. The solution was further circulated for 1 hour, and then it was cooled to room temperature. Thereafter, ethanol was added thereto and the mixture was purified by decantation, and then the sedimentary deposits were dispersed in water to thereby obtain a ZnO nanoparticle (volume average particle diameter: 7 nm) dispersion liquid having a mass concentration of 2.5%. Note that, the volume average particle diameter was measured in the same manner as in Comparative Example 1-1.

To COATSOME EL-01-N manufactured by NOF CORPORATION, 2 mL of a liquid in which the aforementioned ZnO nanoparticle dispersion liquid was diluted to 0.30% by mass with pure water was added, and then the mixture was vibrated to thereby prepare a weakly negatively-charged liposome composition dispersion liquid (ZnO content of 3.0 mg/mL), that was a liposome composition dispersion liquid of Comparative Example 2-1 (hereinafter, may be referred to as Sample 2A).

A volume average dispersed-particle diameter of Sample 2A was measured in the same manner as in Comparative Example 1-1, and it was 250 nm.

Sample 2A was stable in a PBS buffer solution (pH 7.2) (under physiological conditions).

### Example 2-1

Sample 2A obtained in Comparative Example 2-1 was poured into a vial, and the vial was filled with perfluoropropane (PFP) gas. After filling the vial with the gas in the volume that was 1.5 times of the volume of the vial under pressure, ultrasonic waves of 20 kHz and 50 W were applied thereto for 15 minutes. Thereafter, ultrasonic waves of 800 kHz and 30 W were further applied for 60 minutes to thereby obtain a liposome composition dispersion liquid of Example 2-1 (hereinafter, may be referred to as Sample 2B).

A volume average dispersed-particle diameter of Sample 2B was measured in the same manner as in Comparative Example 1-1, and it was 280 nm.

A concentration of the perfluoropropane gas in Sample 2B was determined in the same manner as in Example 1-1, and it was 2.8 μL/mL.

Sample 2B was stable in a PBS buffer solution (pH 7.2).

### Comparative Example 3-1

To 1.62 g of iron (III) chloride hexahydrate, 100 mL of a 20% by mass dextran (molecular weight: 15,000 to 20,000) solution was added, and the mixture was heated at 80° C. to dissolve the contents therein. To this solution, a solution in which 0.63 g of iron (II) chloride tetrahydrate was dissolved in 2.5 mL was added. Into the resulting solution, 6.5 mL of a 14% by mass ammonium water was added by dripping while stirring the solution, so as to neutralize the solution. After the addition of the ammonium water was completed, the solution was stirred and heated at 80° C. for 2 hours, then cooled to room temperature. The cooled solution was subjected to ultrafiltration for desalination purification to remove excess dextran, to thereby obtain a magnetite nanoparticle (volume average particle diameter: 4 nm) dispersion liquid having a mass concentration of 0.65%. Note that, the volume average particle diameter was measured in the same manner as in the Comparative Example 1-1.

To COATSOME EL-01-N manufactured by NOF CORPORATION, 2 mL of a liquid in which the aforementioned magnetite nanoparticle dispersion liquid was diluted to 0.26% by mass with pure water was added, and then the mixture was vibrated to thereby prepare a weakly negatively-charged liposome composition dispersion liquid (magnetite content of 2.6 mg/mL), that was a liposome composition dispersion liquid of Comparative Example 3-1 (hereinafter, may be referred to as Sample 3A).

A volume average dispersed-particle diameter of Sample 3A was measured in the same manner as in Comparative Example 1-1, and it was 280 nm.

Sample 3A was stable in a PBS buffer solution (pH 7.2) (under physiological conditions).

### Example 3-1

Sample 3A obtained in Comparative Example 3-1 was poured into a vial, and the vial was filled with perfluoropropane (PFP) gas. After filling the vial with the gas in the volume that was 1.5 times of the volume of the vial under pressure, ultrasonic waves of 20 kHz and 50 W were applied thereto for 15 minutes. Thereafter, ultrasonic waves of 800 kHz and 30 W were further applied for 60 minutes to thereby obtain a liposome composition dispersion liquid of Example 3-1 (hereinafter, may be referred to as Sample 3B).

A volume average dispersed-particle diameter of Sample 3B was measured in the same manner as in Comparative Example 1-1, and it was 350 nm.

A concentration of the perfluoropropane gas in Sample 3B was determined in the same manner as in Example 1-1, and it was 2.4 μL/mL.

Sample 3B was stable in a PBS buffer solution (pH 7.2).

### Comparative Example 4-1

A SnO₂ aqueous sol (product name: Ceramace C-10, manufacturer: Taki Chemical Co., Ltd., average particle diameter: 2 nm) was subjected to gel-filtration using desalination column (product name: PD10, manufacturer: GE Healthcare Japan K.K.), and the resultant was diluted with pure water to thereby obtain a 0.4% by mass SnO₂ nanoparticle dispersion liquid.

To COATSOME EL-01-N manufactured by NOF CORPORATION, 2 mL of the aforementioned SnO₂ nanoparticle dispersion liquid was added, and then the mixture was vibrated to thereby prepare a weakly negatively-charged liposome composition dispersion liquid (SnO₂ content of 4.0 mg/mL), that was a liposome composition dispersion liquid of Comparative Example 4-1 (hereinafter, may be referred to as Sample 4A).

A volume average dispersed-particle diameter of Sample 4A was measured in the same manner as in Comparative Example 1-1, and it was 230 nm.

Sample 4A was stable in a PBS buffer solution (pH 7.2) (under physiological conditions).

### Example 4-1

Sample 4A obtained in Comparative Example 4-1 was poured into a vial, and the vial was filled with perfluoropropane (PFP) gas. After filling the vial with the gas in the volume that was 1.5 times of the volume of the vial under pressure, ultrasonic waves of 20 kHz and 50 W were applied thereto for 15 minutes. Thereafter, ultrasonic waves of 800 kHz and 30 W were further applied for 60 minutes to thereby obtain a liposome composition dispersion liquid of Example 4-1 (hereinafter, may be referred to as Sample 4B).

A volume average dispersed-particle diameter of Sample 4B was measured in the same manner as in Comparative Example 1-1, and it was 260 nm.

A concentration of the perfluoropropane gas in Sample 4B was determined in the same manner as in Example 1-1, and it was 2.5 μL/mL.

Sample 4B was stable in a PBS buffer solution (pH 7.2).

### Comparative Example 5-1

A ZrO₂ aqueous sol (manufacturer: Sumitomo Osaka Cement Co., Ltd., average particle diameter: 3 nm) was subjected to gel-filtration using desalination column (product name: PD10, manufacturer: GE Healthcare Japan K.K.), and the resultant was diluted with pure water to thereby obtain a 0.4% by mass ZrO₂ nanoparticle dispersion liquid.

To COATSOME EL-01-N manufactured by NOF CORPORATION, 2 mL of the aforementioned ZrO₂ nanoparticle dispersion liquid was added, and then the mixture was vibrated to thereby prepare a weakly negatively-charged liposome composition dispersion liquid (ZrO₂ content of 4.0 mg/mL), that was a liposome composition dispersion liquid of Comparative Example 5-1 (hereinafter, may be referred to as Sample 5A).

A volume average dispersed-particle diameter of Sample 5A was measured in the same manner as in Comparative Example 1-1, and it was 240 nm.

Sample 5A was stable in a PBS buffer solution (pH 7.2) (under physiological conditions).

### Example 5-1

Sample 5A obtained in Comparative Example 5-1 was poured into a vial, and the vial was filled with perfluoropropane (PFP) gas. After filling the vial with the gas in the volume that was 1.5 times of the volume of the vial under pressure, ultrasonic waves of 20 kHz and 50 W were applied thereto for 15 minutes. Thereafter, ultrasonic waves of 800 kHz and 30 W were further applied for 60 minutes to thereby obtain a liposome composition dispersion liquid of Example 5-1 (hereinafter, may be referred to as Sample 5B).

A volume average dispersed-particle diameter of Sample 5B was measured in the same manner as in Comparative Example 1-1, and it was 290 nm.

A concentration of the perfluoropropane gas in Sample 5B was determined in the same manner as in Example 1-1, and it was 2.6 μL/mL. Sample 5B was stable in a PBS buffer solution (pH 7.2).

### Comparative Example 6-1

TiO₂ nanoparticles were made encapsulated in a DTP-DOPE-containing PEG-modified liposomes in the manner described in Example 1, JP-A No. 2005-298486, provided that 6.0 mL of 250 mM ammonium sulfate solution was replaced with 6.0 mL of Sample 1A (0.24% by mass TiO₂ nanoparticle dispersion liquid). Note that, DTP, DOPE, and PEG mentioned above are 3-(2-pyridyldithio)propionitrile, 1,2-dioleyl-sn-glycero-3-phosphoethanol amine, and polyethylene glycol, respectively.

The DTP-DOPE containing PEG-modified liposome composition containing TiO₂ nanoparticles was then bonded to rHSA (genetically-modified human serum albumin) in the manner described in Example 1, JP-A No. 2005-298486 to obtain a TiO₂ nanoparticle-containing PEG-rHSA-modified liposome composition of Comparative Example 6-1 (hereinafter, may be referred to as Sample 6A).

A volume average dispersed-particle diameter of Sample 6A was measured in the same manner as in Comparative Example 1-1, and it was 120 nm.

Sample 6A was stable in a PBS buffer solution (pH 7.2).

### Example 6-1

A liposome composition dispersion liquid of Example 6-1 (hereinafter, may be referred to as Sample 6B) was prepared in the same manner as in Example 1-1, provided that Sample 1A was replaced with Sample 6A.

A volume average dispersed-particle diameter of Sample 6B was measured in the same manner as in Comparative Example 1-1, and it was 150 nm.

A concentration of the perfluoropropane gas in Sample 6B was determined in the same manner as in Example 1-1, and it was 2.8 μL/mL.

Sample 6B was stable in a PBS buffer solution (pH 7.2).

The constitutions of liposome compositions obtained in Examples 1-1 to 6-1, and Comparative Examples 1-1 to 6-1 are summarized in Table 1.

TABLE 1

| | | Metal oxide | | | Gas | | | | | |
|---|---|---|---|---|---|---|---|---|---|---|
| | | | Average | | | (A) | | | | |
| | | | particle | (B) | | Contained | | Liposome | | |
| | | | diameter | Mass | | volume | Concentration | | Dv | Ratio |
| | Sample | Type | (nm) | (mg) | Type | (μL) | (μL/mL) | Type | (nm) | B/A |
| Comp. | 1A | TiO₂ | 6 | 4.8 | — | — | — | COATSOME | 240 | — |
| Ex. 1-1 | | | | | | | | EL-01-N | | |
| Ex. 1-1 | 1B | TiO₂ | 6 | 4.8 | PFP | 5.0 | 2.5 | COATSOME | 270 | 0.96 |
| | | | | | | | | EL-01-N | | |
| Comp. | 1C | — | — | — | — | — | — | COATSOME | 270 | — |
| Ex. 1-2 | | | | | | | | EL-01-N | | |
| Comp. | 1D | — | — | — | PFP | 5.2 | 2.6 | COATSOME | 300 | — |
| Ex. 1-3 | | | | | | | | EL-01-N | | |
| Comp. | 2A | ZnO | 7 | 6.0 | — | — | — | COATSOME | 250 | — |
| Ex. 2-1 | | | | | | | | EL-01-N | | |
| Ex. 2-1 | 2B | ZnO | 7 | 6.0 | PFP | 5.6 | 2.8 | COATSOME | 280 | 1.07 |
| | | | | | | | | EL-01-N | | |
| Comp. | 3A | Magnetite | 4 | 5.2 | — | — | — | COATSOME | 280 | — |
| Ex. 3-1 | | | | | | | | EL-01-N | | |
| Ex. 3-1 | 3B | Magnetite | 4 | 5.2 | PFP | 4.8 | 2.4 | COATSOME | 350 | 1.08 |
| | | | | | | | | EL-01-N | | |
| Comp. | 4A | SnO₂ | 2 | 8.0 | — | — | — | COATSOME | 230 | — |
| Ex. 4-1 | | | | | | | | EL-01-N | | |
| Ex. 4-1 | 4B | SnO₂ | 2 | 8.0 | PFP | 5.0 | 2.5 | COATSOME | 260 | 1.60 |
| | | | | | | | | EL-01-N | | |
| Comp. | 5A | ZrO₂ | 3 | 8.0 | — | — | — | COATSOME | 240 | — |
| Ex. 5-1 | | | | | | | | EL-01-N | | |
| Ex. 5-1 | 5B | ZrO₂ | 3 | 8.0 | PFP | 5.2 | 2.6 | COATSOME | 290 | 1.54 |
| | | | | | | | | EL-01-N | | |
| Comp. | 6A | TiO₂ | 6 | 4.3 | — | — | — | PEG•rHSA | 120 | — |
| Ex. 6-1 | | | | | | | | modified | | |
| | | | | | | | | liposome | | |
| Ex. 6-1 | 6B | TiO₂ | 6 | 4.3 | PFP | 5.6 | 2.8 | PEG•rHSA | 150 | 0.77 |
| | | | | | | | | modified | | |
| | | | | | | | | liposome | | |

In Table 1, “Dv” denotes a volume average dispersed particle diameter.

### Experimental Example 1

### Cancer Cell Killing Test I by Ultrasonic Radiation

Using a human lymphoma cell strain U937, cell-killing effect of each of Samples 1A to 6B was examined by applying ultrasonic wave to each sample.

RPMI 1640 to which 10% FBS had been added was used as a culture solution, and a concentration of cells was adjusted to 1×10⁶ cells/mL. In a 96-well cell culture plate, a cell suspension and the aforementioned sample were both added in an amount of 180 μL and 20 μL, respectively, per well. To this, ultrasonic waves were applied at the intensity of 0.5 W/cm², duty rate of 50% by means of a sonoporator SP-100 (Sonidel Limited) for 10 seconds. After the application of ultrasonic waves, the mixture of the cells and sample was incubated by a CO₂ incubator at 37° C. for 2 hours. Thereafter, a number of living cells was determined and evaluated by a trypan blue-exclusion test. The results are shown in Table 2.

TABLE 2

| | | Number of |
|---|---|---|
| | Sample | living cells |
| Comp. Ex. 1-1 | 1A | 82 |
| Ex. 1-1 | 1B | 38 |
| Comp. Ex. 1-2 | 1C | 102 |
| Comp. Ex. 1-3 | 1D | 79 |
| Comp. Ex. 2-1 | 2A | 75 |
| Ex. 2-1 | 2B | 31 |
| Comp. Ex. 3-1 | 3A | 86 |
| Ex. 3-1 | 3B | 46 |
| Comp. Ex. 4-1 | 4A | 89 |
| Ex. 4-1 | 4B | 58 |
| Comp. Ex. 5-1 | 5A | 90 |
| Ex. 5-1 | 5B | 61 |
| Comp. Ex. 6-1 | 6A | 79 |
| Ex. 6-1 | 6B | 41 |

From the results shown in Table 2, it can be seen that the liposome composition of the present invention in which the liposome entraps the gas, and encapsulates or adsorbs the metal oxide particle(s) had excellent cancer cell killing effects.

### Comparative Example 7-1

A liposome composition dispersion liquid of Comparative Example 7-1 (hereinafter, may be referred to as Sample 7A) was prepared in the same manner as in Comparative Example 1-1, provided that COATSOME EL-01-N was replaced with a mixture of 1,2-distearoyl-sn-glycero-phosphatidylcholine (DSPC) (94 μmol) and 1,2-distearoyl-sn-glycero-3-phosphatidyl-ethanolamine-methoxy-polyethylene glycol (DSPE-PEG) (6 μmol) to form liposomes.

A volume average dispersed-particle diameter of Sample 7A was measured in the same manner as in Comparative Example 1-1, and it was 330 nm.

Sample 7A was stable in a PBS buffer solution (pH 7.2).

### Example 7-1

Sample 7A obtained in Comparative Example 7-1 was poured into a vial, and the vial was filled with perfluoropropane (PFP) gas. After filling the vial with the gas in the volume that was 1.5 times of the volume of the vial under pressure, ultrasonic waves of 20 kHz and 50 W were applied thereto for 15 minutes. Thereafter, ultrasonic waves of 800 kHz and 30 W were further applied for 60 minutes to thereby obtain a liposome composition dispersion liquid of Example 7-1 (hereinafter, may be referred to as Sample 7B).

A volume average dispersed-particle diameter of Sample 7B was measured in the same manner as in Comparative Example 1-1, and it was 390 nm.

A concentration of the perfluoropropane gas in Sample 7B was determined in the same manner as in Example 1-1, and it was 2.7 μL/mL.

Sample 7B was stable in a PBS buffer solution (pH 7.2).

### Example 7-2

A liposome composition dispersion liquid of Example 7-2 (hereinafter, may be referred to as Sample 7C) was prepared in the same manner as in Example 7-1, provided that the perfluoropropane (PFP) gas was replaced with air.

A volume average dispersed-particle diameter of Sample 7C was measured in the same manner as in Comparative Example 1-1, and it was 360 nm.

A concentration of the air in Sample 7C was determined in the same manner as in Example 1-1, and it was 2.0 μL/mL.

Sample 7C was stable in a PBS buffer solution (pH 7.2).

### Example 7-3

A liposome composition dispersion liquid of Example 7-3 (hereinafter, may be referred to as Sample 7D) was prepared in the same manner as in Example 7-1, provided that the perfluoropropane (PFP) gas was replaced with xenon (Xe) gas.

A volume average dispersed-particle diameter of Sample 7D was measured in the same manner as in Comparative Example 1-1, and it was 380 nm.

A concentration of the xenon (Xe) gas in Sample 7D was determined in the same manner as in Example 1-1, and it was 2.3 μL/mL.

Sample 7D was stable in a PBS buffer solution (pH 7.2).

### Example 7-4

A liposome composition dispersion liquid of Example 7-4 (hereinafter, may be referred to as Sample 7E) was prepared in the same manner as in Example 7-1, provided that the perfluoropropane (PFP) gas was replaced with krypton (Kr) gas.

A volume average dispersed-particle diameter of Sample 7E was measured in the same manner as in Comparative Example 1-1, and it was 390 nm.

A concentration of the krypton (Kr) gas in Sample 7E was determined in the same manner as in Example 1-1, and it was 2.5 μL/mL.

Sample 7E was stable in a PBS buffer solution (pH 7.2).

### Example 7-5

A liposome composition dispersion liquid of Example 7-5 (hereinafter, may be referred to as Sample 7F) was prepared in the same manner as in Example 7-1, provided that the perfluoropropane (PFP) gas was replaced with argon (Ar) gas.

A volume average dispersed-particle diameter of Sample 7F was measured in the same manner as in Comparative Example 1-1, and it was 400 nm.

A concentration of the argon (Ar) gas in Sample 7F was determined in the same manner as in Example 1-1, and it was 2.3 μL/mL.

Sample 7F was stable in a PBS buffer solution (pH 7.2).

### Example 7-6

A liposome composition dispersion liquid of Example 7-6 (hereinafter, may be referred to as Sample 7G) was prepared in the same manner as in Example 7-1, provided that the perfluoropropane (PFP) gas was replaced with 1,1,1,2,3,4,4,5,5,5-decafluoropentane.

A volume average dispersed-particle diameter of Sample 7G was measured in the same manner as in Comparative Example 1-1, and it was 350 nm.

A concentration of 1,1,1,2,3,4,4,5,5,5-decafluoropentane in Sample 7G was determined in the same manner as in Example 1-1, and it was 2.6 μL/mL.

Sample 7G was stable in a PBS buffer solution (pH 7.2).

The constitutions of liposome compositions obtained in Examples 7-1 to 7-6 and Comparative Example 7-1 are summarized in Table 3.

TABLE 3

| | | Metal oxide | | | Gas | | | | | |
|---|---|---|---|---|---|---|---|---|---|---|
| | | | Average | | | (A) | | | | |
| | | | particle | (B) | | Contained | | Liposome | | |
| | | | diameter | Mass | | volume | Concentration | | Dv | Ratio |
| | Sample | Type | (nm) | (mg) | Type | (μL) | (μL/mL) | Type | (nm) | B/A |
| Comp. | 7A | TiO₂ | 6 | 4.8 | — | — | — | DSPC + | 330 | — |
| Ex. 7-1 | | | | | | | | DSPE-PEG | | |
| Ex. 7-1 | 7B | TiO₂ | 6 | 4.8 | PFP | 5.4 | 2.7 | DSPC + | 390 | 0.89 |
| | | | | | | | | DSPE-PEG | | |
| Ex. 7-2 | 7C | TiO₂ | 6 | 4.8 | Air | 4.0 | 2.0 | DSPC + | 360 | 1.20 |
| | | | | | | | | DSPE-PEG | | |
| Ex. 7-3 | 7D | TiO₂ | 6 | 4.8 | Xe | 4.6 | 2.3 | DSPC + | 380 | 1.04 |
| | | | | | | | | DSPE-PEG | | |
| Ex. 7-4 | 7E | TiO₂ | 6 | 4.8 | Kr | 5.0 | 2.5 | DSPC + | 390 | 0.96 |
| | | | | | | | | DSPE-PEG | | |
| Ex. 7-5 | 7F | TiO₂ | 6 | 4.8 | Ar | 4.6 | 2.3 | DSPC + | 400 | 1.04 |
| | | | | | | | | DSPE-PEG | | |
| Ex. 7-6 | 7G | TiO₂ | 6 | 4.8 | Decafluoro | 5.2 | 2.6 | DSPC + | 350 | 0.92 |
| | | | | | pentane | | | DSPE-PEG | | |

In Table 3, “Dv” denotes a volume average dispersed particle diameter.

### Experimental Example 2

### Cancer Cell Killing Test II by Ultrasonic Radiation

Using a human cervical cancer cell strain (Hela cells), cell-killing effect of each of Samples 7A to 7G was examined by applying ultrasonic wave to each sample.

MEN to which 10% FBS and 1% NEAA had been added was used as a culture solution, and a concentration of cells was adjusted to 1×10⁵ cells/mL. In a 96-well cell culture plate, a cell suspension and the aforementioned sample were both added in an amount of 180 μL and 20 μL, respectively, per well. To this, ultrasonic waves were applied at the intensity of 1 W/cm², duty rate of 50% by means of a sonoporator SP-100 (Sonidel Limited) for 30 seconds. After the application of ultrasonic waves, the mixture of the cells and sample was incubated by a CO₂ incubator at 37° C. for 2 hours. Thereafter, a number of living cells was determined and evaluated by a trypan blue-exclusion test. The results are shown in Table 4.

TABLE 4

| | | Number of |
|---|---|---|
| | Sample | living cells |
| Comp. Ex. 7-1 | 7A | 95 |
| Ex. 7-1 | 7B | 41 |
| Ex. 7-2 | 7C | 60 |
| Ex. 7-3 | 7D | 49 |
| Ex. 7-4 | 7E | 52 |
| Ex. 7-5 | 7F | 54 |
| Ex. 7-6 | 7G | 48 |

From the results shown in Table 4, it can be seen that the liposome composition of the present invention in which the liposome entraps the gas, and encapsulates or adsorbs the metal oxide particle(s) had excellent cancer cell killing effects.

### Comparative Example 8-1

In the course of preparing the commercial ultrasonic diagnostic contrast agent, SONAZOID for Injection 16 μL (manufactured by Daiichi Sankyo Company, Limited), instead of using the attached water for injection (2 mL), 2 mL of a liquid in which the TiO₂ nanoparticle dispersion liquid prepared in Comparative Example 1-1 was diluted to 0.003% by mass with pure water was added, and the mixture was vibrated to prepare a liposome dispersion liquid (TiO₂ content of 0.03 mg/mL), to thereby obtain a liposome composition dispersion liquid of Comparative Example 8-1 (hereinafter, may be referred to as Sample 8A).

A volume average dispersed-particle diameter of Sample 8A was measured in the same manner as in Comparative Example 1-1, and it was 3.8 μm.

A concentration of the perfluoropropane gas in Sample 8A was determined in the same manner as in Example 1-1, and it was 7.9 μL/mL.

Sample 8A was stable in a PBS buffer solution (pH 7.2).

### Example 8-1

A liposome composition dispersion liquid of Example 8-1 (hereinafter, may be referred to as Sample 8B) was prepared in the same manner as in Comparative Example 8-1, provided that 2 mL of the liquid, in which the TiO₂ nanoparticle dispersion liquid was diluted to 0.003% by mass with pure water, was replaced with 2 mL of a liquid in which the TiO₂ nanoparticle dispersion liquid was diluted to 0.008% by mass with pure water, to prepare a liposome dispersion liquid (TiO₂ content of 0.08 mg/mL).

A volume average dispersed-particle diameter of Sample 8B was measured in the same manner as in Comparative Example 1-1, and it was 3.8 μm.

A concentration of the perfluoropropane gas in Sample 8B was determined in the same manner as in Example 1-1, and it was 7.9 μL/mL.

Sample 8B was stable in a PBS buffer solution (pH 7.2).

### Example 8-2

A liposome composition dispersion liquid of Example 8-2 (hereinafter, may be referred to as Sample 8C) was prepared in the same manner as in Comparative Example 8-1, provided that 2 mL of the liquid, in which the TiO₂ nanoparticle dispersion liquid was diluted to 0.003% by mass with pure water, was replaced with 2 mL of a liquid in which the TiO₂ nanoparticle dispersion liquid was diluted to 0.46% by mass with pure water, to prepare a liposome dispersion liquid (TiO₂ content of 4.6 mg/mL).

A volume average dispersed-particle diameter of Sample 8C was measured in the same manner as in Comparative Example 1-1, and it was 3.8 μm.

A concentration of the perfluoropropane gas in Sample 8C was determined in the same manner as in Example 1-1, and it was 7.9 μL/mL.

Sample 8C was stable in a PBS buffer solution (pH 7.2).

### Example 8-3

A liposome composition dispersion liquid of Example 8-3 (hereinafter, may be referred to as Sample 8D) was prepared in the same manner as in Comparative Example 8-1, provided that 2 mL of the liquid, in which the TiO₂ nanoparticle dispersion liquid was diluted to 0.003% by mass with pure water, was replaced with 2 mL of a liquid in which the TiO₂ nanoparticle dispersion liquid was diluted to 3.9% by mass with pure water, to prepare a liposome dispersion liquid (TiO₂ content of 39 mg/mL).

A volume average dispersed-particle diameter of Sample 8D was measured in the same manner as in Comparative Example 1-1, and it was 3.6 μm.

A concentration of the perfluoropropane gas in Sample 8D was determined in the same manner as in Example 1-1, and it was 7.9 μL/mL.

Sample 8D was stable in a PBS buffer solution (pH 7.2).

### Comparative Example 8-2

A liposome composition dispersion liquid of Comparative Example 8-2 (hereinafter, may be referred to as Sample 8E) was prepared in the same manner as in Comparative Example 8-1, provided that 2 mL of the liquid, in which the TiO₂ nanoparticle dispersion liquid was diluted to 0.003% by mass with pure water, was replaced with 2 mL of a liquid in which the TiO₂ nanoparticle dispersion liquid was condensed to 4.6% by mass, to prepare a liposome dispersion liquid (TiO₂ content of 46 mg/mL).

A volume average dispersed-particle diameter of Sample 8E was measured in the same manner as in Comparative Example 1-1, and it was 3.3 μm.

A concentration of the perfluoropropane gas in Sample 8E was determined in the same manner as in Example 1-1, and it was 7.8 μL/mL.

Sample 8E tended to precipitate in a PBS buffer solution (pH 7.2).

The constitutions of liposomes obtained in Examples 8-1 to 8-3 and Comparative Examples 8-1 to 8-2 are summarized in Table 5.

TABLE 5

| | | Metal oxide | | | Gas | | | Liposome | | | |
|---|---|---|---|---|---|---|---|---|---|---|---|
| | | | Average | | | (A) | | | | Stability | |
| | | | particle | (B) | | Contained | | | | under | |
| | | | diameter | Mass | | volume | Concentration | | Dv | physiological | |
| | Sample | Type | (nm) | (mg) | Type | (μL) | (μL/mL) | Type | (nm) | conditions | Ratio B/A |
| Commercial | SONAZOID | — | — | — | Perfluoro | 15.8 | 7.9 | hydrogenated egg | 3.8 | Stable | — |
| product | | | | | butane | | | phosphatidyl | | | |
| | | | | | | | | serine sodium | | | |
| | | | | | | | | salt | | | |
| Comp. | 8A | TiO₂ | 6 | 0.06 | Perfluoro | 15.8 | 7.9 | hydrogenated egg | 3.8 | Stable | 0.004 |
| Ex. 8-1 | | | | | butane | | | phosphatidyl | | | |
| | | | | | | | | serine sodium | | | |
| | | | | | | | | salt | | | |
| Ex. 8-1 | 8B | TiO₂ | 6 | 0.16 | Perfluoro | 15.8 | 7.9 | hydrogenated egg | 3.8 | Stable | 0.01 |
| | | | | | butane | | | phosphatidyl | | | |
| | | | | | | | | serine sodium | | | |
| | | | | | | | | salt | | | |
| Ex. 8-2 | 8C | TiO₂ | 6 | 9.2 | Perfluoro | 15.8 | 7.9 | hydrogenated egg | 3.8 | Stable | 0.58 |
| | | | | | butane | | | phosphatidyl | | | |
| | | | | | | | | serine sodium | | | |
| | | | | | | | | salt | | | |
| Ex. 8-3 | 8D | TiO₂ | 6 | 78 | Perfluoro | 15.8 | 7.9 | hydrogenated egg | 3.6 | Stable | 4.94 |
| | | | | | butane | | | phosphatidyl | | | |
| | | | | | | | | serine sodium | | | |
| | | | | | | | | salt | | | |
| Comp. | 8E | TiO₂ | 6 | 92 | Perfluoro | 15.6 | 7.8 | hydrogenated egg | 3.3 | Precipitated | 5.90 |
| Ex. 8-2 | | | | | butane | | | phosphatidyl | | | |
| | | | | | | | | serine sodium | | | |
| | | | | | | | | salt | | | |

In Table 5, “Dv” denotes a volume average dispersed particle diameter.

### Experimental Example 3

### Growth Inhibition Test of Melanoma on Mice by Ultrasonic Radiation

Female nude mice of 5 weeks old were used for the test, and 100 μL of melanoma cells (C32 cells) adjusted to 2×10⁷ cell (cell viability≧98%) was hypodermically injected to each mouse. When the tumor was grown to have the diameter of approximately 5 mm, a treatment was started. For a treatment, the mice were randomly separated into 7 groups (5 mice in each group), for six different treatments including an ultrasonic treatment only, SONAZOID with an ultrasonic treatment, and each of Samples 8A to 8E with an ultrasonic treatment. While giving the mice inhalation anesthesia, 10 μL of the sample was locally injected to the mice of each group, and ultrasonic waves were applied thereto at a frequency of 1 MHz, intensity of 1 W/cm², and duty ratio of 50% for 2 minutes by means of a sonoporation SONITRON 1000 (manufactured by Rich-Mar Corp.). For comparison, 5 mice whose tumors were not treated were also provided.

The injection of the sample and ultrasonic radiation were both performed every other day, 5 times in total, and the size of the tumor (represented as a product of the long axis and the short axis) was measured in two weeks after the last treatment. The results are shown in Table 6. It was found that Sample 8E tended to precipitate in the blood, and the sufficient fluidity thereof could not be attained.

TABLE 6

| | | Size of tumor (cm²) |
|---|---|---|
| | Sample | (average of 5 mice) |
| Commercial | SONAZOID | 125 |
| product | | |
| Comp. | 8A | 122 |
| Ex. 8-1 | | |
| Ex. 8-1 | 8B | 98 |
| Ex. 8-2 | 8C | 66 |
| Ex. 8-3 | 8D | 61 |
| Comp. | 8E | 64 |
| Ex. 8-2 | | |
| Ultrasonic | — | 128 |
| only | | |
| No | — | 150 |
| treatment | | |

From the results shown in Tables 5 and 6, it can be seen that the liposome composition of the present invention, in which the liposome entraps the gas therein, and encapsulates or adsorbs metal oxide particles therein or thereon and the ratio B/A is 0.01 to 5 where A is the volume (μL) of the gas contained and B is the mass (mg) of the metal oxide particles, is stably dispersed under physiological conditions, and the liposome composition of the present invention exhibits an effect of inhibiting the growth of melanoma on mice so that it is effective as a therapeutic enhancer.

### Experimental Example 4

### Liver Cancer Cystography Test on Rats by Ultrasonic Radiation

Cancer cells were implanted to rats in advance, and 10 μL of each of SONAZOID and Samples 8A to 8D was injected to a tail vein of each rat. After a certain period, an ultrasonography was performed by a harmonic method (TOSHIBA Ultrasound Aplio 80 (manufactured by Toshiba Medical Systems Corporation)). As a result, Samples 8A to 8D provided the same degree of accuracy and contrast in the obtained image of the liver cancer to that with SONAZOID. It was also found that Sample 8E could not secure its fluidity in the blood, and thus it could not provide a contrasted image.

Accordingly, it was found that the liposome composition of the present invention in which the liposome entraps the air thereof, and encapsulates or adsorbs TiO₂ therein or thereon was effective as a diagnostic contrast agent.

The liposome composition of the present invention in which the liposome entraps the gas therein, and encapsulate or adsorbs the metal oxide particle(s) therein or thereon has excellent dispersion stability in an aqueous medium in the neutral pH range, and is suitably used, for example, as a diagnostic contrast agent, therapeutic enhancer, and pharmaceutical composition, which are used for diagnoses and therapies mainly using ultrasonic waves.

Moreover, since the liposome composition of the present invention can accurately visualize the distribution of the gas by an ultrasonic diagnostic equipment, a treatment can be carried out at the same time as highly accurately detecting a lesioned part such as cancer. Therefore, the liposome composition of the present invention contributes to a quality of life (QOL) of a patient.

What is claimed is:

1. A liposome composition, comprising:
at least one liposome;
gas entrapped in the liposome; and
at least one metal oxide particle encapsulated in or adsorbed on the liposome,
wherein the liposome composition satisfies a ratio B/A of 0.01 to 5, where A is a volume of the gas contained in the liposome on the basis of micro liter, and B is a mass of the at least one metal oxide particle contained in the liposome on the basis of milligram.

2. The liposome composition according to claim 1, wherein the liposome composition has a volume average dispersed-particle diameter of 20 nm to 20 μm.

3. The liposome composition according to claim 1, wherein the gas is at least one selected from the group consisting of oxygen, nitrogen, carbon dioxide, xenon, krypton, argon, hydrofluorocarbons, and perfluorocarbons.

4. The liposome composition according to claim 1, wherein the at least one metal oxide particle has a volume average particle diameter of 1 nm to 50 nm.

5. The liposome composition according to claim 1, wherein the metal oxide particle is a particle of metal oxide, which is at least one selected from the group consisting of titanium oxide, zinc oxide, iron oxide, tin oxide, and zirconium oxide.

6. The liposome composition according to claim 1, further comprising a receptor bonded to or contained in the liposome, wherein the receptor is capable of specifically recognizing a certain tissue.

7. The liposome composition according to claim 1, wherein the liposome composition is ultrasonic sensitive.

8. The liposome composition according to claim 1, wherein the liposome composition is used for medical purposes.

9. A diagnostic contrast agent, comprising: a liposome composition,
wherein the liposome composition comprises:
at least one liposome;
gas entrapped in the liposome; and
at least one metal oxide particle encapsulated in or adsorbed on the liposome,
wherein the liposome composition satisfies a ratio B/A of 0.01 to 5, where A is a volume of the gas contained in the liposome on the basis of micro liter, and B is a mass of the at least one metal oxide particle contained in the liposome on the basis of milligram.

10. A therapeutic enhancer, comprising: a liposome composition,
wherein the liposome composition comprises:
at least one liposome;
gas entrapped in the liposome; and
at least one metal oxide particle encapsulated in or adsorbed on the liposome,
wherein the liposome composition satisfies a ratio B/A of 0.01 to 5, where A is a volume of the gas contained in the liposome on the basis of micro liter, and B is a mass of the at least one metal oxide particle contained in the liposome on the basis of milligram.

11. A pharmaceutical composition, comprising: a liposome composition,
wherein the liposome composition comprises:
at least one liposome;
gas entrapped in the liposome; and
at least one metal oxide particle encapsulated in or adsorbed on the liposome,
wherein the liposome composition satisfies a ratio B/A of 0.01 to 5, where A is a volume of the gas contained in the liposome on the basis of micro liter, and B is a mass of the at least one metal oxide particle contained in the liposome on the basis of milligram.
